(19) United States
(12) Patent Application Publication
(10) Pub. No.: US 2019/0328640 A1
(43) Pub. Date: Oct. 31, 2019

(54) Process for Relaxing Curls and/or Straightening Keratin Fibres, Using a Composition Low in Reducing Agents and Straightening Kit

(71) Applicant: L'OREAL, Paris (FR)

(72) Inventors: Henri Samain, Bievres (FR); Virginie Burckbuchler, Aulnay-Sous-Bois (FR)

(73) Assignee: L'OREAL, Paris (FR)

(21) Appl. No.: 16/472,828

(22) PCT Filed: Dec. 22, 2017
(86) PCT No.: PCT/EP2017/084295

§ 371 Date: Jun. 21, 2019

(30) Foreign Application Priority Data
Dec. 22, 2016 (FR) 1663186

(51) Int. Cl. A61K 8/46; A61K 8/34; A61K 8/368; A61Q 5/04

(52) U.S. Cl. CPC A61K 8/46; A61K 8/347; A61K 2800/30; A61Q 5/04; A61K 2800/884; A61K 8/368

(57) ABSTRACT

The present invention relates to a process for relaxing curls and/or straightening keratin fibres, such as the hair, which comprises the application to the fibres of one or more compositions comprising reducing agents, and a step of heat treatment of the fibres by means of a heating tool. A subject of the invention is also the use of the composition(s) comprising reducing agents in a process for relaxing curls and/or straightening keratin fibres. Finally, a subject of the invention is a multi-compartment device or “kit” suitable for carrying out such a process.

The present invention relates to a process for relaxing curls and/or straightening keratin fibres, such as the hair, which comprises the application to the fibres of one or more compositions comprising reducing agents, and a step of heat treatment of the fibres by means of a heating tool.

A subject of the invention is also the use of the composition(s) comprising reducing agents in a process for relaxing curls and/or straightening keratin fibres.

Finally, a subject of the invention is a multi-compartment device or “kit” suitable for carrying out such a process.

Many people are not satisfied by the appearance of their hair. In particular, people who have curly hair usually seek to obtain straight hair.

To obtain permanent reshaping of the hair such as straightening of the hair, uncurling or relaxing of curls, the technique most commonly used consists, in a first stage, in opening the —S—S— disulfide bonds of keratin (keratocystine) by means of a generally basic composition containing a sulfur-comprising reducing agent (reduction step), and then, after having rinsed the head of hair thus treated, generally with water, in reconstituting, in a second stage, said disulfide bonds by applying to the hair, which has been placed under tension beforehand, an oxidizing composition (oxidation step, also known as the fixing step) so as finally to give the hair the desired shape.

The new shape given to the hair by such a chemical treatment is eminently long-lasting and especially withstands washing with water or shampoos, as opposed to the simple standard techniques of temporary reshaping, such as hairsetting.

Many products intended for straightening or uncurling the hair or for relaxing curls exist on the market.

The products intended for straightening or uncurling are generally formulated using thiols and alkaline agents, whereas the products intended for relaxing curls instead contain products of the cysteine type and derivatives thereof.

The oxidizing compositions required for performing the fixing step are usually compositions based on aqueous hydrogen peroxide solution.

These products are generally applied to curly or voluminous hair in order to obtain more or less pronounced straightening, a reduction in volume and in mass of the head of hair.

However, this technique is not entirely satisfactory. The application of these products is generally long, with a longer or shorter leave-on time depending on the product, the type of hair and the desired effect. It requires a precise know-how, which is mainly due to the high contents of reducing agents used in the reducing compositions or to the high contents of hydroxides and/or to the very alkaline pH of uncurling compositions, and also to the various more or less long leave-on times of these compositions.

Furthermore, reducing agents are generally used in high concentrations, which may lead to more or less pronounced degradation of the keratin fibre, in particular when the hair is dyed.

This technique can thus create, in the long term, a detrimental modification of the quality of the hair, leading to a decrease in its cosmetic properties, such as its vitality or its sheen, and a degradation in its mechanical properties, more particularly in its mechanical strength. These drawbacks are in particular observed with thioglycolic acid which is generally used in the basic medium at pH values ranging from 8.5 to 9.5.

In addition, the use of such a straightening treatment is a choice that is generally drastic for the consumer, who cannot adjust the level of straightening according to the desired result.

Moreover, it has also been found that the use of these reducing agents or of these strong alkaline agents can lead to scalp discomfort (irritation, itching, etc.).

Finally, the compositions employed often pose problems of odours, in particular the reducing compositions and especially those containing thiols. Hair treated with these compositions can also retain an unpleasant odour.

There is therefore a real need to develop treatments for shaping and/or straightening keratin fibres, in particular the hair, that are gentler than those which exist in the prior art, and that do not have all of the drawbacks described above, that is to say that they do not modify the integrity and the quality of keratin fibres, while at the same time providing the keratin fibres with good cosmetic properties.

In parallel, this process must retain good performance levels in terms of straightening keratin fibres, in particular with an effect that is remanent with respect to several shampooing operations.

Furthermore, it is desirable for these treatments to be easy to apply, and to allow a modulation of the straightening effect, for example by having recourse to repeated applications, while at the same time reducing the odours resulting from the implementation of a such a process.

The applicant has now discovered that the implementation of a particular process in at least two steps makes it possible to meet the abovementioned objectives.

It has thus been discovered, surprisingly, a process for shaping and/or straightening of keratin fibres, in particular human keratin fibres such as the hair, in several steps, which comprises:
i) the application to said fibres of an acidic composition (A), preferably having a pH of inclusively between 1 and 5 and containing one or more thiol-comprising reducing agents;
ii) the application to said fibres of a distinct composition (B) containing one or more non-thiol-comprising reducing agents; followed
iii) by a step of heat treatment of the fibres by means of a heating tool;
   it being understood that steps i) and ii) are carried out separately i) then ii) or else ii) then i). Preferably, the steps of the process are carried out according to the following order i) then ii) then iii).

This process makes it possible to achieve the desired properties, inter alia in terms of integrity, of quality and of cosmeticity of the keratin fibres, while at the same time obtaining shaping and/or straightening of keratin fibres of good and long-lasting quality.

A subject of the present invention is thus a process for shaping and/or straightening keratin fibres, in particular keratin fibres such as the hair, comprising the steps i), ii) and iii) as defined previously.

The implementation of this process makes it possible to obtain shaping and/or straightening of keratin fibres which is of good quality and is remanent with respect to several shampooing operations, while at the same time preserving the quality and the integrity of the keratin fibres. Furthermore, the implementation of this method makes it possible to provide the keratin fibres with good cosmetic properties, in particular sheen and softness to the touch.

It has also been found that the process according to the invention makes it possible to boost or exacerbate the color of the keratinous fibers, in particular it makes it possible to obtain a better colour in terms of colour uptake and/or chromaticity of the hair.

In one preferred variant, the process according to the invention is a process for straightening keratin fibres, in particular the hair.

A subject of the invention is also a kit suitable for implementing the process of the invention. This kit comprises at least two compartments:
a first compartment comprising a first acidic composition (A), preferably at a pH of inclusively between 1 and 5, which comprises i) one or more thiol-comprising reducing agents; and
a second compartment comprising a second composition (B) which comprises ii) one or more non-thiol-comprising reducing agents.

A subject of the present invention is also a composition (A) suitable for implementing the process of the invention. This cosmetic composition comprises one or more thiol-comprising reducing agents, is acidic and has a pH of inclusively between 1 and 5, preferably between 2.5 and 4.

The term “pH of inclusively between 1 and 5” is understood to mean that the limits 1 and 5 are included in the pH range.

A subject of the present invention is also the use of the compositions (A) and (B) in a process for, shaping, relaxing curls and/or straightening keratin fibres, in particular human keratin fibres such as the hair.

Such a process is rapid and simple to perform, and does not require any particular know-how.

Furthermore, the implementation of this process makes it possible to obtain a relaxing of curls and/or a reduction in the volume of the head of hair, which is produced gradually. By virtue of its protection of the integrity of keratin fibres, the implementation of this process makes it possible to give them good cosmetic properties, in particular of colouration, sheen and softness to the touch.

In particular, it is possible to modulate the desired effect, by successive implementations of such a process.

Finally, the process according to the invention makes it possible to substantially reduce the uncomfortable odours resulting from the implementation of a conventional process for relaxing curls and/or straightening.

Other characteristics and advantages of the invention will emerge more clearly on reading the description, the examples and the figures that follow.

For the purposes of the present invention and unless otherwise indicated:
the term “(hetero)aryl” is intended to mean aryl and heteroaryl groups;
the “aryl” or “heteroaryl” radicals or the aryl or heteroaryl part of a radical may be substituted with at least one substituent borne by a carbon atom, chosen from:
   C₁-C₆ and preferably C₁-C₄ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl or thiol group;
   a C₁-C₆ alkoxy or C₁-C₆ alkylthio radical;
   a (poly)hydroxy(C₂-C₆)alkoxy radical;
   an amino radical;
   a 5- or 6-membered heterocycloalkyl radical, preferentially morpholino, piperazino, piperidino or pyrolidino, which is optionally substituted with a (C₁-C₄) alkyl radical, preferentially methyl;
   a 5- or 6-membered heteroaryl radical, preferentially imidazolyl, optionally substituted with a (C₁-C₄)alkyl radical, preferentially methyl;
   an amino radical substituted with one or two identical or different C₁-C₆ alkyl radicals, optionally bearing at least:
      i) a hydroxyl group,
      ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
      iii) a quaternary ammonium group —N⁺R′R″R′″, M⁻ for which R′, R″ and R′″, which may be identical or different, represent a hydrogen atom or a C₁-C₄ alkyl group and M⁻ represents an anionic counterion,
      iv) or an optionally cationic 5- or 6-membered heteroaryl radical, preferentially imidazolium, optionally substituted with a (C₁-C₄)alkyl radical, preferentially methyl;
   an acylamino radical (—N(R)—C(O)—R′) in which the R radical is a hydrogen atom or a C₁-C₄ alkyl radical optionally bearing at least one hydroxyl group and the R′ radical is a C₁-C₂ alkyl radical;
   a carbamoyl radical ((R)₂N—C(O)—) in which the R radicals, which may be identical or different, represent a hydrogen atom or a C₁-C₄ alkyl radical optionally bearing at least one hydroxyl group;
   an alkylsulfonylamino radical (R′—S(O)₂—N(R)—) in which the R radical represents a hydrogen atom or a C₁-C₄ alkyl radical optionally bearing at least one hydroxyl group and the R′ radical represents a C₁-C₄ alkyl radical, or a phenyl radical;
   an amino sulfonyl radical ((R)₂N—S(O)₂—) in which the R radicals, which may be identical or different, represent a hydrogen atom or a C₁-C₄ alkyl radical optionally bearing at least one hydroxyl group;
   a carboxyl radical in the acid or salified form (preferably salified with an alkali metal or a substituted or unsubstituted ammonium);
   a cyano group;
   a nitro or nitroso group;
   a polyhaloalkyl group, preferably trifluoromethyl;
   the cyclic or heterocyclic part of a non-aromatic radical may be substituted with at least one substituent chosen from the following groups:
   hydroxyl;
   C₁-C₄ alkoxy, C₂-C₄ (poly)hydroxyalkoxy;
   C₁-C₄ alkyl;
   alkylcarbonylamino (R—C(O)—N(R′)—) in which the radical R′ is a hydrogen atom or a C₁-C₄ alkyl radical optionally bearing at least one hydroxyl group, and the radical R is a C₁-C₂ alkyl radical or an amino radical optionally substituted with one or two C₁-C₄ alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
alkylcarbonyloxy (R—C(O)—O—) in which the radical R is a C₁-C₄ alkyl radical or an amino group optionally substituted with one or two identical or different C₁-C₄ alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
alkoxycarbonyl (R—X—C(O)—) in which the radical R is a C₁-C₄ alkoxy radical, X is an oxygen atom or an amino group optionally substituted with a C₁-C₄ alkyl group itself optionally bearing at least one hydroxyl group, said alkyl radical possibly forming with the nitrogen atom to which it is attached a saturated or unsaturated, optionally substituted 5- to 7-membered heterocycle, optionally comprising at least one other nitrogen or non-nitrogen heteroatom;
a cyclic or heterocyclic radical, or a non-aromatic part of an aryl or heteroaryl radical, may also be substituted with one or more oxo groups;
a hydrocarbon-based chain is unsaturated when it comprises one or more double bonds and/or one or more triple bonds;
an “aryl” radical represents a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl;
a “heteroaryl radical” represents a fused or non-fused, optionally cationic, 5- to 22-membered monocyclic or polycyclic group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl, xanthinyl or thioxanthinyl;
a “heterocyclic radical” is a fused or non-fused, 5- to 22-membered monocyclic or polycyclic non-aromatic radical, possibly containing one or two unsaturations and comprising from 1 to 6 heteroatoms chosen from nitrogen, oxygen, sulfur and selenium;
an “alkyl” radical is a linear or branched, saturated C₁-C₁₀, in particular C₁-C₈, more particularly C₁-C₆ and preferably C₁-C₄, hydrocarbon-based radical;
an “alkoxy” radical is an “alkyl-oxy” radical in which the alkyl part is as defined previously;
an “alkenyl” radical is a linear or branched C₂-C₁₀, in particular C₂-C₈, more particularly C₂-C₆, preferably C₂-C₄, hydrocarbon-based radical comprising one or more conjugated or non-conjugated unsaturations, preferably comprising one or two double bonds, such as ethylenyl;
the expression “optionally substituted” attributed to the alkyl or alkenyl radical is intended to mean that said alkyl or alkenyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom; v) carboxy; vi) or aryl such as phenyl optionally substituted with one or more (di)(C₁-C₄)(alkyl)amino groups or hydroxyl groups;
an “alkoxy” radical is an alkyl-oxy radical for which the alkyl radical is a linear or branched C₁-C₈ and preferentially C₁-C₆ hydrocarbon-based radical;
when the alkoxy group is optionally substituted, this implies that the alkyl group is optionally substituted as defined above;
   the term “organic or mineral acid salt” is more particularly intended to mean salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)—OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)—OH; xiv) triflic acid CF₃SO₃H; and xv) tetrafluoroboric acid HBF₄;
   the term “anionic counterion” is intended to mean an anion or an anionic group derived from an organic or mineral acid which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) carboxylates Alk-C(O)—OH with Alk representing a (C₁-C₆)alkyl group optionally substituted with one or more hydroxyl or carboxylate groups such as citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻, xiii) phosphates O═P(OH)₂—O⁻, O═P(O⁻)₂—OH O═P(O⁻)₃, HO—[P(O)(O⁻)]_{w}—P(O)(O⁻)₂ with w being an integer; xiv) acetate; xv) triflate; and xvi) borates such as tetrafluoroborate, and xvii) disulfate (O=)₂S(O⁻)₂ or SO₄²⁻ and monosulfate HSO₄⁻.

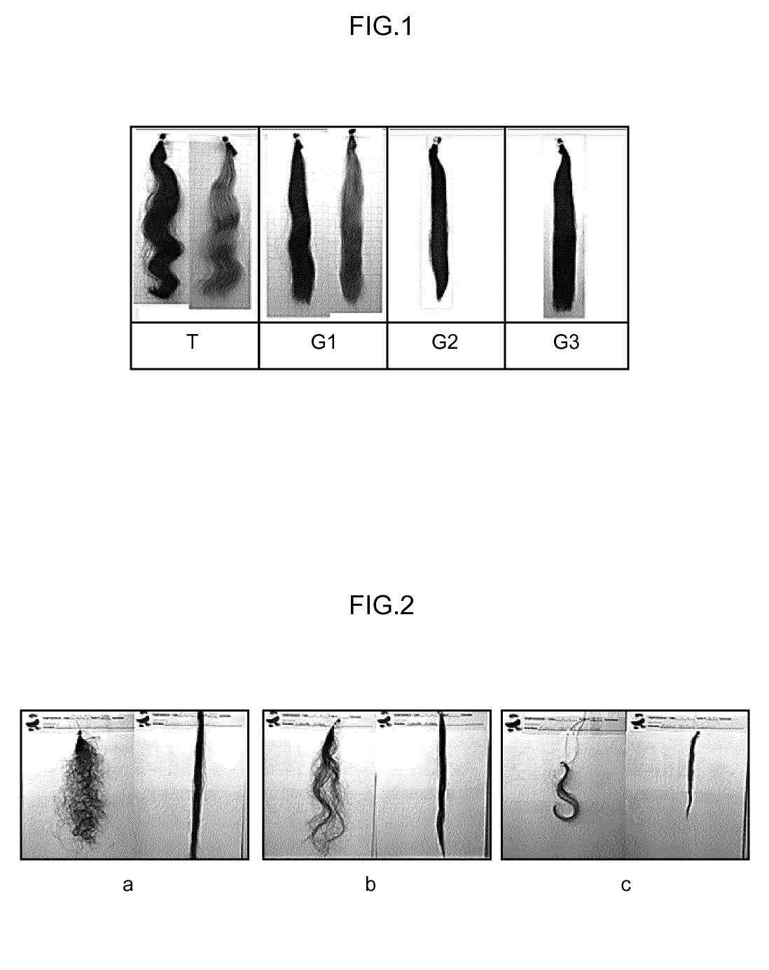

FIG. 1 shows locks of untreated hair (T), or treated with a solution of thiolactic acid at a concentration of 8% (pH=3.5) (G1) or treated with a composition containing either 1% of thiolactic acid and 5% of para-hydroxyphenol at pH=3.5 (G2), or 4% of thiolactic acid and 1% of para-hydroxyphenol at pH=3.5 (G3).

FIG. 2 shows hair treated using the protocol of the invention with a composition containing 1% of thiolactic acid and 5% of para-hydroxyphenol at pH=3.5. The samples a, b and c correspond to the hair of three different volunteers.

As previously explained, the process according to the invention uses i) at least one thiol-comprising reducing agent and ii) at least one non-thiol-comprising reducing agent, it being understood that the composition(s) containing said thiol-comprising reducing agent(s) is (are) acids, preferably said composition(s) has (have) a pH of inclusively between 1 and 5, preferably of inclusively between 2.5 and 4.

The thiol-comprising reducing agent(s) present in the composition (A) used according to the invention are chosen from organic compounds comprising one or more mercapto (—SH or —S—) groups, or disulfide (—S—S—) groups, preferably —SH groups and at least one other function chosen from carboxylic acid, amine, amide, ester and alcohol functions and mixtures thereof.

According to one particular embodiment of the invention, the reducing agent(s) used in the invention are chosen from those of formulae i-1 and i-2, and also the organic or mineral acid or base salts thereof, optical isomers thereof and tautomers thereof, and the solvates such as hydrates:

R—SH i-1

R′—S—R″ i-2

In which formulae i-1 and i-2:
R represents:
   a linear or branched (C₁-C₈) alkyl group, preferably (C₁-C₆) alkyl group,
   a) which is optionally substituted, preferably substituted, with one or more groups chosen from carboxy C(O)OH, (di)(C₁-C₄)(alkyl)amino, hydroxyl —OH and thiol —SH, and/or
   b) optionally interrupted with one or more heteroatoms or groups chosen from —O—, —S—, —N(R′″)— wherein R′″ represents a hydrogen atom or a linear or branched (C₁-C₄)alkyl group, C(O) or combinations thereof such as —O—C(O)—, —C(O)—O—, —N(R′″)—C(O)— or —C(O)—N(R′″)—;
   a (hetero)aryl group optionally substituted in particular with one or more hydroxyl, thiol or carboxy groups;
R′ and R″, which may be identical or different, represent a (C₁-C₈)alkyl group, preferably (C₁-C₆)alkyl group, substituted with one or more groups chosen from hydroxyl, thiol and carboxy;
or else R′ and R″ form, together with the sulfur atom which bears them, a heterocyclic group, comprising from 5 to 7 ring members, which is preferably saturated, which comprises from 1 to 3 heteroatoms, and which is optionally substituted (in particular with one or more (C₁-C₆)alkyl groups optionally substituted with one or more hydroxyl, thiol or carboxy groups), more preferentially the heterocyclic group is a dithiolane group optionally substituted with a (C₁-C₆)alkyl group optionally substituted with one or more carboxy groups.

According to one particular embodiment of the invention, the reducing agents are of formula i-1, in particular those for which R represents a linear or branched (C₁-C₈)alkyl group, preferably (C₁-C₆)alkyl group, substituted with one or more groups chosen from carboxy C(O)OH, amino, hydroxyl —OH, and thiol —SH;

and/or optionally interrupted with one or more heteroatoms or groups chosen from —O—, —N(R′″)— wherein R′″ represents a hydrogen atom or a linear or branched (C₁-C₄)alkyl group, C(O) or combinations thereof such as —O—C(O)—, —C(O)—O—, —N(R′″)—C(O)— or —C(O)—N(R′″)—. Preferably, R represents a linear or branched, uninterrupted (C₁-C₈)alkyl group, preferably (C₁-C₆)alkyl group.

According to another particular embodiment of the invention, the reducing agents are of formula i-1 for which R represents:
a phenyl group optionally substituted with one or more hydroxyl, thiol or carboxy groups; or
a heteroaryl comprising from 5 to 10 ring members, which is preferably bicyclic comprising 9 or 10 ring members, comprising from 1 to 4 heteroatoms chosen from O, S or N, preferably N, optionally substituted with one or more hydroxyl or thiol groups.

According to another particular embodiment of the invention, the reducing agents are of formula i-2, in particular those for which R′ and R″, which may be identical or different, represent a (C₁-C₈)alkyl group, preferably (C₁-C₆)alkyl group, substituted with one or more groups chosen from hydroxyl, thiol and carboxy.

According to another particular embodiment of the invention, the reducing agents are of formula i-2, in particular those for which R′ and R″ form, together with the sulfur atom which bears them, a heterocyclic group, comprising from 5 to 7 ring members, which is preferably saturated, which comprises from 1 to 3 heteroatoms, and which is optionally substituted with one or more (C₁-C₆)alkyl groups optionally substituted with one or more hydroxyl, thiol or carboxy groups, more preferentially the heterocyclic group is a dithio lane group optionally substituted with a (C₁-C₆)alkyl group optionally substituted with one or more hydroxyl, thiol or carboxy groups.

Preferably, the reducing agent(s) comprising at least one mercapto or disulfide group of the invention are chosen from thioglycolic acid, thiolactic acid or 2-mercaptopropionic acid, cysteine, cysteamine, homocysteine, glutathione, thioglycerol, thiomalic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, in particular glyceryl monothioglyco late, and mixtures of these compounds.

The thiol-comprising reducing agent(s) as defined previously may be used in particular in the form of salts, in particular alkali metal salts such as sodium and potassium salts, alkaline-earth metal salts, for example magnesium and calcium salts, ammonium salts, amine salts and amino alcohol salts. Ammonium thioglycolate may thus be used as thiol.

Particularly preferably, the thiol-comprising reducing agent(s) is (are) chosen from thioglycolic acid and salts thereof, thio lactic acid and salts thereof, cysteamine and salts thereof, and mixtures thereof.

Even more preferentially, the thiol-comprising reducing agent(s) is (are) chosen from thioglycolic acid and thio lactic acid.

The thiol-comprising reducing agent(s) included in the composition (A) according to the invention is (are) preferably present in an amount ranging from 0.02% to 5% by weight, preferably from 0.1% to 3% by weight, relative to the total weight of said composition.

The pH of the composition (A) and/or (B) according to the invention may be adjusted to the desired value by means of basifying agents or acidifying agents that are customarily used.

The organic alkaline agent(s) is (are) preferably chosen from alkanolamines, in particular mono-, di- ou tri-hydroxy(C₁-C₆)alkylamine, such as triethanolamine, oxyethylenated and/or oxypropylenated ethylenediamines, amino acids, polyamines of formula (VIII) below, and mixtures thereof:

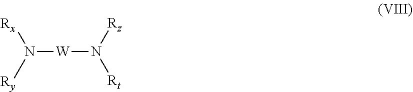

In which formula (VIII) W is a divalent C₁-C₆ alkylene radical optionally substituted with one or more hydroxyl groups or a C₁-C₆ alkyl radical, and/or optionally interrupted with one or more heteroatoms such as O, or —NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ amino alkyl radical.

Examples of amines of formula (VIII) that may be mentioned include 1,3-diaminopropane, 1,3-diamino-2-propanol, spermine and spermidine.

The term “alkanolamine” is intended to mean an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that may be used are of natural or synthetic origin, in their L, D or racemic form, and comprise at least one acid function chosen more particularly from carboxylic acid, sulfonic acid, phosphonic acid and phosphoric acid functions. The amino acids may be in neutral or ionic form.

Among the basifying agents, mention may more particularly be made of aqueous ammonia, alkanolamines, and mineral or organic hydroxides.

Among the acidifying agents, mention may be made of i) hydrochloric acid HCl, ii) hydrobromic acid HBr, iii) sulfuric acid H₂SO₄, iv) alkylsulfonic acids: Alk-S(O)₂OH, such as methanesulfonic acid and ethanesulfonic acid; v) arylsulfonic acids: Ar—S(O)₂OH, such as benzenesulfonic acid and toluenesulfonic acid; vi) (poly)hydroxylated carboxylic acids, such as citric acid, succinic acid, tartaric acid or lactic acid, vii) alkoxysulfinic acids: Alk-O—S(O)—OH, such as methoxysulfinic acid and ethoxysulfinic acid; viii) aryloxysulfinic acid, such as tolueneoxysulfinic acid and phenoxysulfinic acid; ix) phosphoric acid H₃PO₄; x) acetic acid CH₃C(O)—OH; xi) triflic acid CF₃SO₃H and xii) tetrafluoroboric acid HBF₄; more particularly, the mineral or organic acids used to acidify the composition(s) are chosen from hydrochloric acid, orthophosphoric acid, sulfuric acid, carboxylic acids, for instance acetic acid, tartaric acid, citric acid and lactic acid, and sulfonic acids.

The concentration of pH-adjusting agent(s) is in particular adjusted according to the pH of 1 to 5, preferentially 2.5 to 4, designed for the composition(s) containing the thiol-comprising reducing agent(s).

According to one particular embodiment of the invention, the non-thiol-comprising reducing agent(s) present in the composition (B) according to the invention is (are) chosen from ortho-diphenol derivatives having reducing properties. In a manner known per se, the term “ortho-diphenol” denotes compounds comprising at least one aromatic ring, preferably a benzene ring, comprising at least two hydroxyl (OH) groups borne by two adjacent carbon atoms of the aromatic ring which in addition do not comprise a mercapto or disulfide group.

The aromatic ring may more particularly be a fused aryl or fused heteroaromatic ring, i.e. optionally comprising one or more heteroatoms, such as benzene, naphthalene, tetrahydronaphthalene, indane, indene, anthracene, phenanthrene, isoindole, indoline, isoindoline, benzofuran, dihydrobenzofuran, chroman, isochroman, chromene, isochromene, quinoline, tetrahydroquinoline and isoquino line, said aromatic ring comprising at least two hydroxyl groups borne by two adjacent carbon atoms of the aromatic ring. Preferentially, the aromatic ring of the ortho-diphenol derivatives according to the invention is a benzene ring.

The term “fused ring” is intended to mean that at least two saturated or unsaturated and heterocyclic or non-heterocyclic rings have a common bond, i.e. that at least one ring is joined side by side with another ring.

The ortho-diphenols according to the invention may or may not be salified. They can also be in the aglycone form (without bonded sugar) or in the form of glycosylated compounds.

More particularly, the ortho-diphenol derivative represents a compound of formula (I), or an oligomer thereof, in salified or non-salified form:

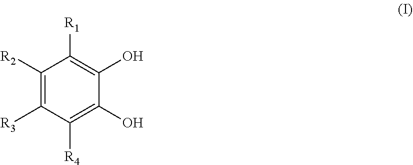

in which formula (I) the substituents:
R₁ to R₄, which may be identical or different, represent:
a hydrogen atom,
a halogen atom,
a hydroxyl radical,
a carboxyl radical;
an alkyl carboxylate or alkoxycarbonyl radical,
an optionally substituted amino radical,
an optionally substituted and linear or branched alkyl radical,
an optionally substituted and linear or branched alkenyl radical,
an optionally substituted cycloalkyl radical,
an alkoxy radical,
an alkoxyalkyl radical,
an alkoxyaryl radical, it being possible for the aryl group to be optionally substituted,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
or two of the substituents carried by two adjacent carbon atoms R₁-R₂, R₂-R₃ or R₃-R₄ form, together with the carbon atoms carrying them, a saturated or unsaturated and aromatic or non-aromatic ring, optionally comprising one or more heteroatoms and optionally fused with one or more saturated or unsaturated rings optionally comprising one or more heteroatoms. Particularly, R₁ to R₄ together form from one to four rings.

A specific embodiment of the invention relates to ortho-diphenol derivatives of formula (I), two adjacent substituents R₁-R₂, R₂-R₃ or R₃-R₄ of which cannot form, with the carbon atoms which carry them, a pyrrolyl radical. More particularly, R₂ and R₃ cannot form a pyrrolyl radical fused to the benzene ring bearing the two hydroxyls.

The ortho-diphenols of use in the process of the invention can be natural or synthetic. The natural ortho-diphenols include the compounds which may be present in nature and which are reproduced by chemical (semi)synthesis.

The salts of the ortho-diphenols of the invention can be salts of acids or of bases. The acids can be mineral or organic. Preferably, the acid is hydrochloric acid, which results in chlorides.

The bases can be mineral or organic. In particular, the bases are alkali metal hydroxides, such as sodium hydroxide, which results in sodium salts.

According to one particular embodiment of the invention, the composition(s) comprise(s) as ingredient one or more synthetic ortho-diphenol derivative(s) that do (does) not exist in nature.

According to another preferred embodiment of the invention, the process for shaping and/or straightening keratin fibres uses, as non-thiol-comprising agent, one or more natural ortho-diphenol derivative(s).

More particularly, the ortho-diphenols that may be used in the process of the invention are in particular:
flavanols, such as catechin and epicatechin gallate,
flavonols, such as quercetin,
anthocyanidins, such as cyanidin, delphinidin or petunidin,
anthocyanins or anthocyans, for instance myrtillin,
ortho-hydroxybenzoates, for example gallic acid salts,
flavones, such as luteolin,
hydroxystilbenes, for example 3,3′,4,5′-tetrahydroxystilbene, optionally oxylated (for example glucosylated),
3,4-dihydroxyphenylalanine and the derivatives thereof,
2,3-dihydroxyphenylalanine and the derivatives thereof,
4,5-dihydroxyphenylalanine and the derivatives thereof,
dihydroxycinnamates, such as caffeic acid and chlorogenic acid,
ortho-polyhydroxycoumarins,
ortho-polyhydroxyisocoumarins,
ortho-polyhydroxycoumarones,
ortho-polyhydroxyisocoumarones,
ortho-polyhydroxychalcones,
ortho-polyhydroxychromones,
ortho-polyhydroxyquinones,
ortho-polyhydroxyxanthones,
1,2-dihydroxybenzene and the derivatives thereof,
1,2,4-trihydroxybenzene and the derivatives thereof,
1,2,3-trihydroxybenzene and the derivatives thereof,
2,4,5-trihydroxytoluene and the derivatives thereof,
proanthocyanidins and especially the proanthocyanidins A1, A2, B1, B2, B3 and C1,
proanthocyanins,
tannic acid,
ellagic acid,
and the mixtures of the preceding compounds.

When the dyeing precursors exhibit D and L forms, both forms can be used in the compositions according to the invention, as can the racemates.

According to one embodiment, the natural ortho-diphenols result from extracts of animals, bacteria, fungi, algae or plants, used in their entirety or partially. In particular as regards plants, the extracts are derived from plants or plant parts, such as fruit, including citrus fruit, vegetables, trees or shrubs. Use may also be made of mixtures of these extracts rich in ortho-diphenols as defined above.

Preferably, the natural ortho-diphenol(s) of the invention is (are) derived from plants or plant parts.

The extracts are obtained by extraction from various plant parts, such as, for example, the root, the wood, the bark, the leaf, the flower, the fruit, the seed, the pod or the peel.

Use may also be made of mixtures of plant extracts.

According to a specific embodiment of the invention, the ortho-diphenol derivative(s) are natural extracts rich in ortho-diphenols. According to a preferred form, the ortho-diphenol derivative(s) are solely natural extracts.

The natural extracts according to the invention may be in the form of powders or liquids. Preferably, the extracts of the invention are provided in the form ofpowders.

According to one particular embodiment of the invention, the non-thiol-comprising reducing agent(s) present in the composition (B) according to the invention is (are) chosen from meta-hydroxyphenol derivatives, also called resorcinols, having reducing properties. In a manner known per se, the term “resorcinols” denotes compounds which comprise at least one aromatic ring, preferably a benzene ring, comprising at least two hydroxyl (OH) groups borne by two carbon atoms which are in the meta-position with respect to one another, and which also do not comprise a mercapto or disulfide group.

The aromatic ring may more particularly be a fused aryl or fused heteroaromatic ring, i.e. optionally comprising one or more heteroatoms, such as benzene, naphthalene, tetrahydronaphthalene, indane, indene, anthracene, phenanthrene, isoindole, indoline, isoindoline, benzofuran, dihydrobenzofuran, chroman, isochroman, chromene, isochromene, quinoline, tetrahydroquinoline and isoquinoline, said aromatic ring comprising at least two hydroxyl groups borne by two carbon atoms which are in the meta-position with respect to one another. Preferentially, the aromatic ring of the resorcinol derivatives according to the invention is a benzene ring.

According to one more particular embodiment of the invention, the non-thiol-comprising reducing agent(s) present in the composition(s) used according to the invention is (are) chosen from meta-hydroxyphenol derivatives of formula (II), and also the organic or mineral acid or base salts thereof, and the solvates thereof such as hydrates:

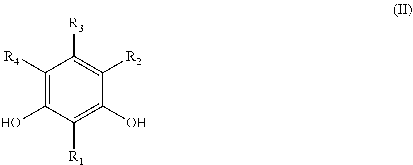

in which formula (II) the substituents:
R₁, R₂ and R₄, which may be identical or different, represent:
a hydrogen atom,
a halogen atom,
a carboxyl radical,
an alkyl carboxylate or alkoxycarbonyl radical,
an optionally substituted amino radical,
an optionally substituted and linear or branched alkyl radical,
an optionally substituted and linear or branched alkenyl radical,
an optionally substituted cycloalkyl radical,
an alkylcarbonyl radical,
a carboxaldehyde radical,
an alkoxy radical,
an alkoxyalkyl radical,
an alkoxyaryl radical, it being possible for the aryl group to be optionally substituted,
an arylalkylcarbonyl radical of which the aryl group, particularly phenyl group, is optionally substituted, preferably with one or more hydroxyl groups,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
R₃ represents:
a hydrogen atom,
a halogen atom,
a hydroxyl radical,
a carboxyl radical,
an alkyl carboxylate or alkoxycarbonyl radical,
an optionally substituted amino radical,
an optionally substituted and linear or branched alkyl radical,
a linear or branched alkenyl radical which is optionally substituted, in particular with a phenyl group which is preferably optionally substituted with one or more (di)(C₁-C₄)(alkyl)amino, or hydroxyl groups,
an optionally substituted cycloalkyl radical,
an alkylcarbonyl radical,
a carboxaldehyde radical,
an alkoxy radical,
an alkoxyalkyl radical,
an alkoxyaryl radical, it being possible for the aryl group to be optionally substituted,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
or two of the substituents borne by two adjacent carbon atoms R₂-R₃ or R₃-R₄ form, together with the carbon atoms carrying them, a saturated or unsaturated and non-aromatic ring, optionally comprising one or more heteroatoms and optionally fused with one or more saturated or unsaturated rings optionally comprising one or more heteroatoms. Particularly, R₂ to R₄ together form from two to four rings.

More particularly, the non-thiol-comprising reducing agent(s) present in the composition (B) according to the invention is (are) chosen from meta-hydroxyphenols of formula (II) in which the substituents:
R₁, R₃ and R₄, which may be identical or different, represent:
   a hydrogen atom,
   a halogen atom,
   a carboxyl radical,
   an alkyl carboxylate or alkoxycarbonyl radical,
   an optionally substituted amino radical,
   an optionally substituted and linear or branched alkyl radical,
   a linear or branched alkenyl radical which is optionally substituted, in particular with a phenyl group which is preferably optionally substituted with one or more (di)(C₁-C₄)(alkyl)amino, or hydroxyl groups,
   an alkylcarbonyl radical,
   a carboxaldehyde radical,
   an alkoxy radical, and/or
R₂ represents:
   a hydrogen atom,
   a halogen atom,
   a hydroxyl radical,
   a carboxyl radical,
   an alkyl carboxylate or alkoxycarbonyl radical,
   an optionally substituted amino radical,
   a phenylalkylcarbonyl radical of which the phenyl group is optionally substituted, preferably with one or more hydroxyl groups,
   an optionally substituted and linear or branched alkyl radical,
   an optionally substituted and linear or branched alkenyl radical,
   an alkylcarbonyl radical,
   a carboxaldehyde radical,
   an alkoxy radical.

More particularly, the substituent R₂ in formula (II) represents a hydrogen atom or a (C₁-C₆)alkyl group, preferably a hydrogen atom.

According to one particular embodiment of the invention, the non-thiol-comprising reducing agent(s) present in the composition(s) used according to the invention is (are) chosen from resorcinol derivatives of formula (II), and also the organic or mineral acid or base salts thereof, and the solvates thereof such as hydrates: in which R¹ to R⁴, which may be identical or different, represent a hydrogen or halogen atom, or a group chosen from:
optionally substituted (C₁-C₁₀)alkyl, in particular optionally substituted with at least one hydroxyl radical;
optionally substituted (C₂-C₁₀)alkenyl, in particular optionally substituted with at least one aryl group, such as a phenyl group, which is optionally substituted with one or more (di)(C₁-C₄)(alkyl)amino, or hydroxyl groups;
(C₁-C₁₀)alkoxy;
carboxy —C(O)—OH or carboxylate —C(O)—O—, M+; with M+ representing a cationic counterion such as an alkali metal or alkaline-earth metal, or an ammonium;
ester —C(O)—O—R⁵ or —O—C(O)—R⁵, with R⁵ representing a (C₁-C₁₀)alkyl group, particularly —C(O)—O—R⁵;
amido —C(O)—NR⁶R⁷ or —NR⁶—C(O)—R⁷ with R⁶ and R⁷, which may be identical or different, representing a hydrogen atom or a (C₁-C₁₀)alkyl group, particularly —C(O)—NH₂;
(C₁-C₁₀)alkylcarbonyl;
hydroxyl; and
amino —NR⁸R⁹, with R⁸ and R⁹, which may be identical or different, representing a hydrogen atom or a group (C₁-C₁₀)alkyl, particularly —NH₂.

In particular, in formula (II), R₁, R₂ and R₄ represent a hydrogen atom or an optionally substituted (C₁-C₆)alkyl group, preferably hydrogen, and/or R₃ represents a hydrogen atom or a linear or branched (C₁-C₆)alkyl or (C₂-C₆)alkenyl group, such as ethanyl, optionally substituted with an aryl group, such as a phenyl group, which is optionally substituted, preferably with one or more (di)(C₁-C₄)(alkyl)amino, or hydroxyl groups.

More preferentially, the non-thiol-comprising reducing agent(s) present in the composition(s) used according to the invention is (are) chosen from the resorcinol derivative of formula (II), in which R₁, R₂ and R₄ represent a hydrogen atom or a (C₁-C₄)alkyl group, preferably hydrogen, and R₃ represents a hydrogen atom or an Ar—CH═CH— group with Ar representing an aryl group, in particular a phenyl group, said aryl group being optionally substituted, preferably with one or more hydroxyl groups; in particular said hydroxyl group(s) is (are) substituted in the ortho or para position with respect to the phenyl group.

Preferably, the meta-hydroxyphenols according to the invention do not comprise two hydroxyl groups borne by two adjacent carbons.

The meta-hydroxyphenols of the invention may be natural or synthetic. Among the natural meta-hydroxyphenols are compounds that may be present in nature and that are reproduced by chemical (semi)synthesis. The salts of the meta-hydroxyphenols of the invention can be salts of acids or of bases. The acids can be mineral or organic. Preferably, the acid is hydrochloric acid, which results in chlorides. The bases can be mineral or organic. In particular, the bases are alkali metal hydroxides, such as sodium hydroxide, which results in sodium salts.

According to a particular embodiment of the invention, the composition comprises, as ingredient a), one or more synthetic meta-hydroxyphenol(s) derivative(s) that do not exist in nature.

According to one embodiment, the natural meta-hydroxyphenols are derived from extracts of animals, bacteria, fungi, algae, plants and fruits, used in their entirety or partially. In particular regarding plants, the extracts are derived from fruits, including citrus fruits, from vegetables, from trees and from shrubs. Use may also be made of mixtures of these extracts, which are rich in meta-hydroxyphenols as defined above.

According to one particular embodiment of the invention, the non-thiol-comprising reducing agent(s) present in the composition (B) according to the invention is (are) chosen from para-hydroxyphenol derivatives having reducing properties. In a manner known per se, the term “para-hydroxyphenol” denotes compounds which comprise at least one aromatic ring, preferably a benzene ring, comprising at least two hydroxyl (OH) groups borne by two carbon atoms which are in the para-position with respect to one another, and which also do not comprise a mercapto or disulfide group.

According to one more particular embodiment of the invention, the non-thiol-comprising reducing agent(s) present in the composition(s) used is (are) chosen from para-hydroxyphenol derivatives of formula (III), and also the organic or mineral acid or base salts thereof, and the solvates thereof such as hydrates:

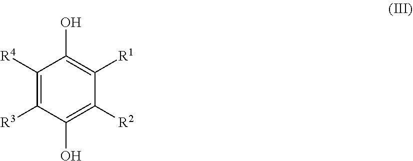

in which formula (III) R¹ to R⁴, which may be identical or different, are as defined previously for formulae (I) and (II), preferably represent a hydrogen atom or an optionally substituted (C₁-C₄)alkyl group, preferably hydrogen.

Preferentially, the non-thiol-comprising agent(s) is (are) chosen from catechol, gallic acid, para-hydroxyphenol or resveratrol, it being understood that, when the aromatic ring of the ortho diphenols, meta hydroxyphenols or para-hydroxyphenols bear more than 2 hydroxyl groups (for example 3, 4, etc.), the compounds should be understood according to the following rule:

If 3 hydroxyl groups are adjacent on the aromatic ring: (position 1, 2, 3 for example), then said non-thiol-comprising reducing agent will be considered to be an ortho-diphenol.

If there are 3 hydroxyl groups of which two are adjacent on the aromatic ring and one is opposite (positions 1, 2, 4 or 1, 2, 5), then said non-thiol-comprising reducing agent will be considered to be a para-hydroxyphenol.

If there are 3 hydroxyl groups of which none is adjacent (position 1, 3, 5), then said non-thiol-comprising reducing agent will be considered to be a meta-hydroxyphenol.

Preferentially, the non-thiol-comprising reducing agent(s) represent(s) from 1% to 10% by weight and preferably from 2% to 8% by weight relative to the total weight of each composition containing it (them).

Preferably, the weight ratio between the amount of thiol-comprising reducing agent(s) and the amount of non-thiol-comprising reducing agent(s) is between 0.01 and 10, particularly between inclusively 0.1 and 5, and more preferentially between inclusively 0.2 and 1 in the composition containing them.

In one embodiment, the pH of the composition (B) is between inclusively 1 and 5, preferably between 2.5 and 4.

In one embodiment, the pH of the composition (A) is between inclusively 1 and 5, preferably between 2.5 and 4.

According to one preferred embodiment of the invention, the cosmetic composition (A) does not comprise hydrogen peroxide, more preferably the composition (A) of the invention does not comprise any chemical oxidizing agent other than oxygen in the air.

According to another preferred embodiment of the invention, the cosmetic composition (B) does not comprise hydrogen peroxide, more preferably the composition (B) of the invention does not comprise any chemical oxidizing agent other than oxygen in the air.

In one particular embodiment of the invention, the composition (B) comprising the non-thiol-comprising agent(s) is generally applied before and/or after the composition (A) comprising the thiol-comprising agent(s), and preferably after the composition (A). In other words, the process according to the invention comprises step i) of applying the composition (A), ii) then the step of applying the composition (B), then iii) the heating step. This alternative is preferred.

According to one preferred embodiment, the composition(s) used according to the invention are non-colouring, that is to say that they do not comprise direct or oxidation dying agents other than the non-thiol-comprising reducing agents when they are coloured.

In this preferred embodiment, the process according to the invention preferably comprises a step of rinsing the keratin fibres after step i) and/or ii) and before step iii) of heat treatment.

Most particularly preferably in this embodiment, the process comprises:
the application to the fibres of a composition (A) comprising said thiol-comprising agent(s) at a pH of between 1 and 5, then
the application to the fibres of a composition (B) comprising said non-thiol-comprising agent(s), then
an optional step of rinsing the fibres, then
a step of heat treatment of the fibres by means of a heating tool.

According to one particular embodiment of the invention, the composition(s) used in the process according to the invention can also comprise one or more non-ionic, anionic, cationic, or amphoteric or zwitterionic surfactants.

### Surfactants

The composition according to the present invention may optionally also comprise one or more surfactants.

The surfactant(s) that may be used in the composition according to the invention may be chosen from non-ionic surfactants, cationic surfactants, anionic surfactants and amphoteric or zwitterionic surfactants, and mixtures thereof.

The composition according to the present invention may thus comprise one or more non-ionic surfactants.

The non-ionic surfactants that may be used are described, for example, in the Handbook of Surfactants by M. R. Porter, published by Blackie & Son (Glasgow and London), 1991, pp. 116-178.

Examples of non-ionic surfactants that may be mentioned include the following non-ionic surfactants:
oxyalkylenated (C₈-C₂₄)alkylphenols;
saturated or unsaturated, linear or branched, oxyalkylenated or glycerolated C₈-C₄₀ alcohols, comprising one or two fatty chains;
saturated or unsaturated, linear or branched, oxyalkylenated C₈-C₃₀ fatty acid amides;
esters of saturated or unsaturated, linear or branched, C₈-C₃₀ acids and of polyethylene glycols;
preferably oxyethylenated esters of saturated or unsaturated, linear or branched, C₈-C₃₀ acids and of sorbitol;
esters of fatty acids and of sucrose;
(C₈-C₃₀)alkyl(poly)gluco sides, (C₈-C₃₀)alkenyl(poly)gluco sides, which are optionally oxyalkylenated (0 to 10 oxyalkylene units) and comprising from 1 to 15 glucose units, (C₈-C₃₀)alkyl(poly)glucoside esters;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
aldobionamides;
amine oxides;
oxyethylenated and/or oxypropylenated silicones;
and mixtures thereof.

The oxyalkylene units are more particularly oxyethylene or oxypropylene units, or their combination, preferably oxyethylene units.

The number of moles of ethylene oxide and/or of propylene oxide preferably ranges from 1 to 250, more particularly from 2 to 100 and better still from 2 to 50; the number of moles of glycerol ranges in particular from 1 to 50 and better still from 1 to 10.

Advantageously, the non-ionic surfactants according to the invention do not comprise any oxypropylene units.

As examples of glycerolated non-ionic surfactants, use is preferably made of monoglycerolated or polyglycerolated C₈-C₄₀ alcohols, comprising from 1 to 50 mol of glycerol and preferably from 1 to 10 mol of glycerol.

Preference is more particularly given, among the glycerolated alcohols, to the use of the C₈/C₁₀ alcohol comprising 1 mol of glycerol, the C₁₀/C₁₂ alcohol comprising 1 mol of glycerol and the C₁₂ alcohol comprising 1.5 mol of glycerol.

The non-ionic surfactant(s) that may be used in the dye composition according to the invention are preferentially chosen from:
oxyethylenated C₈ to C₄₀ alcohols comprising from 1 to 100 mol of ethylene oxide, preferably from 2 to 50 and more particularly from 2 to 40 mol of ethylene oxide and comprising one or two fatty chains;
saturated or unsaturated oxyethylenated vegetable oils comprising from 1 to 100 and preferably from 2 to 50 mol of ethylene oxide;
(C₅-C₃₀)alkyl(poly)glucosides, which are optionally oxyalkylenated (0 to 10 OE) and comprising 1 to 15 glucose units;
monoglycerolated or polyglycerolated C₈ to C₄₀ alcohols, comprising from 1 to 50 mol of glycerol and preferably from 1 to 10 mol of glycerol;
saturated or unsaturated, linear or branched, oxyalkylenated C₅-C₃₀ fatty acid amides;
esters of saturated or unsaturated, linear or branched, C₅-C₃₀ acids and of polyethylene glycols;
and mixtures thereof.

The composition according to the present invention can comprise one or more cationic surfactants.

“Cationic surfactant” is understood to mean a surfactant which is positively charged when it is present in the compositions according to the invention. This surfactant can carry one or more permanent positive charges or can contain one or more cationizable functional groups within the compositions according to the invention.

The cationic surfactant or surfactants are preferably chosen from primary, secondary or tertiary fatty amines which are optionally polyoxyalkylenated, or their salts, quaternary ammonium salts, and mixtures thereof.

The fatty amines generally comprise at least one C₈ to C₃₀ hydrocarbon-based chain.

As quaternary ammonium salts, mention may in particular be made of those corresponding to the general formula below:

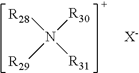

in which the groups R₂₈ to R₃₁, which may be identical or different, represent a linear or branched aliphatic group containing from 1 to 30 carbon atoms, or an aromatic group such as aryl or alkylaryl, at least one of the groups R₂₈ to R₃₁ denoting a group containing from 8 to 30 carbon atoms, preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups, X⁻ is an anion chosen from the group of halides, phosphates, acetates, lactates, (C₁-C₄)alkyl sulfates, and (C₁-C₄)alkyl- or (C₁-C₄)alkylarylsulfonates.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The composition according to the present invention may comprise one or more anionic surfactants.

The term “anionic surfactant” is intended to mean a surfactant comprising, as ionic or ionizable groups, only anionic groups. These anionic groups are preferably chosen from the groups —COOH, —COO—, —SO₃H, —SO₃⁻, —OSO₃H, —OSO₃⁻, —PO₂H₂, —PO₂H⁻, —PO₂²⁻, —P(OH)₂, ═P(O)OH, —P(OH)O—, ═P(O)O—, ═POH and ═PO⁻, the anionic parts comprising a cationic counterion such as an alkali metal, an alkaline-earth metal or an ammonium.

The composition according to the present invention can comprise one or more amphoteric or zwitterionic surfactants.

In particular, the amphoteric or zwitterionic surfactant(s), which are preferably non-silicone, which may be used in the composition according to the present invention may in particular be derivatives of optionally quaternized, secondary or tertiary aliphatic amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

The surfactants that can be used in the process according to the invention are preferably non-ionic or cationic.

When they are present, the surfactant(s) preferably represent from 0.1% to 20% by weight and better still from 1% to 10% by weight relative to the total weight of each composition containing it (them).

The composition(s) that can be used in the process according to the invention generally comprise(s) water, which typically represents from 10% to 90% by weight, preferably from 10% to 80% by weight, preferably from 10% to 70% by weight, relative to the total weight of each composition.

The compositions that can be used in the process according to the invention may also contain cosmetically acceptable organic solvents, more particularly including alcohols such as ethyl alcohol, isopropyl alcohol, benzyl alcohol and phenylethyl alcohol, or non-aromatic polyols or polyol ethers, for instance ethylene glycol monomethyl, monoethyl or monobutyl ethers, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

The solvents may then represent from 0.5% to 20% by weight and preferably from 2% to 10% by weight relative to the total weight of each composition containing them.

According to one preferred embodiment of the invention, the process for shaping and/or straightening keratin fibres does not use hydrogen peroxide, more preferably the process according to the invention does not use any chemical oxidizing agent other than oxygen of the air.

The compositions used according to the invention may also comprise one or more cosmetic adjuvants other than the compounds described previously.

For example, they may comprise one or more standard additive(s) that are well known in the art, such as linear or cyclic, volatile or non-volatile silicones, cationic, non-ionic, anionic or amphoteric polymers, peptides and derivatives thereof, protein hydrolysates, waxes, agents for preventing hair loss, vitamins and provitamins including panthenol, sunscreens, mineral or organic pigments, sequestrants, plasticizers, solubilizers, mineral or organic thickeners, antioxidants, nacreous agents, fragrances and preservatives.

Needless to say, those skilled in the art will take care to select this or these optional additional compound(s) such that the advantageous properties intrinsically associated with the compositions used according to the invention are not, or are not substantially, adversely affected by the envisaged addition(s).

The above additives may generally be present in an amount, for each of them, of between 0 and 20% by weight relative to the total weight of the composition containing them.

Those skilled in the art can choose the appropriate formulation form for the compositions according to the invention, and also their methods of preparation, on the basis of their general knowledge, taking into account first the nature of the constituents used, in particular their solubility in the support, and secondly the application envisaged for the composition. Thus, the composition(s) according to the invention may be in the form of a suspension or a dispersion, in particular of oil-in-water by means of vesicles; an optionally thickened or even gelled oily solution; an oil-in-water, water-in-oil or multiple emulsion; a gel or a mousse; an oily or emulsified gel; a dispersion of vesicles, in particular lipid vesicles; a two-phase or multiphase lotion; a spray. These compositions can have the appearance of a lotion, a cream, a salve, a soft paste, an ointment, a solid that has been cast or moulded in particular as a stick or in a dish, or a compacted solid.

The compositions used in the process according to the invention can thus be in any form compatible with an application to keratin fibres, for example in the form of a wax, a paste, a more or less fluid or thick cream, gel, foam, a spray or a lotion.

The compositions described previously are applied to dry or wet keratin fibres, sequentially or simultaneously.

The compositions are usually left in place on the fibres for a time generally ranging from 1 minute to 1 hour and preferably from 5 minutes to 30 minutes for each composition.

As described previously, the process according to the invention comprises a step of heat treatment of the fibres by means of a heating tool.

This heat treatment step is generally carried out following the application of the composition(s) described above, optionally after elimination thereof by rinsing.

Preferably, the heating tool is chosen from a hairstyling hood, a straightening iron (flat iron), a hairdryer and an infrared-ray dispenser, and more preferentially the heating tool is a straightening iron. The iron is applied by successive separate strokes lasting a few seconds or by gradual movement or sliding along the locks.

The hair treatment step is generally carried out at a temperature ranging from 30 to 250° C., preferably from 60 to 230° C. and more preferentially from 100 to 150° C.

According to one particular embodiment of the invention, the process for treating keratin fibres comprises, as heat treatment, a step of smoothing/uncurling the keratin fibres by means of a heating tool chosen from irons and a steam iron, i.e. “irons” which comprise a device that emits steam and that applies this steam before, during or after the straightening/uncurling.

The term “iron” is intended to mean, within the meaning of the present invention, a device for heating keratin fibres, said fibres and the heating device being brought into contact. The end of the iron which comes into contact with the keratin fibres generally exhibits two flat surfaces. These two surfaces can be made of metal or of ceramic. In particular, these two surfaces can be smooth or crimped or curved.

According to a particular embodiment, the iron or the steam iron is at a temperature of between 65° C. and 250° C., particularly between 80° C. and 230° C., more particularly greater than or equal to 100° C. and preferentially between 100° C. and 190° C. Preferably, the heat treatment step of the process for treating keratin fibres is carried out at a temperature ranging from 150° C. to 230° C., preferably ranging from 160° C. to 230° C., preferentially ranging from 160° C. to 210° C., especially ranging from 180° C. to 200° C.

As examples of irons that may be used in the straightening process according to the invention, mention may be made of any type of steam flat iron, and in particular, nonlimitingly, those described in U.S. Pat. Nos. 5,957,140 and 5,046,516.

The steam iron may be applied by successive separate strokes lasting a few seconds or by gradual movement or sliding along the tresses of keratin fibres, especially of hair.

Preferably, the steam iron is applied in the process according to the invention with a continuous movement from the root to the tip of the hair, in one or more passes, in particular in two to twenty passes. The duration of each pass of the steam iron may range from 2 seconds to 1 minute.

Advantageously, steam is applied to keratin fibres, especially the hair, according to a flow rate of less than 5 g/min, especially of between 1 and 4 g/min.

The application of steam may be performed using any machine known per se for generating the amount of steam of use in the process of the invention. According to a particular embodiment, this machine is portable, i.e. the tank for generating steam is in contact with the part of the device comprising the steam-dispensing orifices. The steam application step may be performed before, during or after the heating step, and preferably before.

Preferably, the step of straightening/relaxing the keratin fibres is carried out for a time that may range from 2 seconds to 30 minutes, and preferentially from 2 seconds to 20 minutes, better still from 2 seconds to 10 minutes, better still from 2 seconds to 5 minutes and even better still from 2 seconds to 2 minutes.

Preferably, step ii) is carried out with a steampod steam straightener device.

The process according to the invention may also comprise an additional step of drying the keratin fibres, after the application step of i), and of ii), and before the heat treatment. The drying step can be carried out using a hand-held hairdryer or a hood dryer or by drying in the open air. The drying step is advantageously performed at a temperature ranging from 20 to 70° C.

After the step of straightening/relaxing using the steam iron, the keratin fibres may be optionally rinsed with water or washed with a shampoo. The keratin fibres are subsequently optionally dried using a hand-held hairdryer or a hood dryer or by drying in the open air.

According to one embodiment, the process according to the invention is carried out on natural keratin fibres, in particular natural hair.

According to another embodiment, the process according to the invention is carried out on damaged keratin fibres, in particular damaged hair. The term “damaged hair” is intended to mean dry or rough or brittle or limp hair or hair with split ends. The process of the invention is particularly carried out on sensitized human keratin fibres, in particular sensitized hair, such as bleached, uncurled or permanent-waved fibres.

The process according to the invention may be carried out on keratin fibres, in particular hair, which is dry or wet. Preferentially, the process is carried out on natural keratin fibres.

After the application steps of i) and ii) of the process of the invention, and before the heat treatment step iii) is carried out, the composition(s) A and/or B applied can be left for a period ranging from 1 to 60 minutes, preferably ranging from 2 to 50 minutes, preferentially ranging from 5 to 45 minutes. The leave-on time may take place at a temperature ranging from 15° C. to 45° C., preferably at ambient temperature (25° C.).

The cosmetic composition which contains the thiol-comprising reducing agents, as described previously, is advantageously applied to the keratin fibres in an amount ranging from 0.1 to 10 grams and preferably from 0.2 to 5 grams of composition per gram of keratin fibres.

The cosmetic composition which contains the non-thiol-comprising reducing agents, as described previously, is advantageously applied to the keratin fibres in an amount ranging from 0.1 to 10 grams and preferably from 0.2 to 5 grams of composition per gram of keratin fibres.

After application of the cosmetic composition(s) A and/or B to the keratin fibres, the latter may be wrung out to remove the excess composition or washed with water or with a shampoo.

The treatment process according to the invention may be carried out before, during and/or after an additional process of cosmetic treatment of the keratin fibres, such as a process for temporary shaping (shaping with curlers, a crimping iron or a straightening iron) or a process for long-lasting shaping (permanent-waving or uncurling) of the keratin fibres.

The process according to the invention may also comprise an additional step of partially pre-drying the hair fibres before the step of increasing the temperature, so as to prevent significant amounts of steam being given off, which might burn the stylist's hands and the subject's scalp. This pre-drying step can be carried out for example by means of a dryer or of a hood or else by drying in the open air.

Before and/or at the end of the process according to the invention, the keratin fibres may optionally be washed with a shampoo, followed by rinsing with water, before being dried or left to dry.

The process according to the invention can be repeated in order to increase the effects, until the desired level of straightening is obtained. However, and this constitutes an advantage of the present invention; from the very first implementation of the process according to the invention, including an implementation without placing the hair under tension, a substantial reduction in the volume of the head of hair is observed. When the hair is curly, relaxation of the curls and/or better curl definition are also observed.

It is also found that the unpleasant odours that are given off during the straightening process, or that remain on the straightened hair, are reduced by means of performing the process according to the invention.

According to one preferred embodiment, the process is repeated several times, either consecutively, or after a delay ranging from a few hours to a few days.

A subject of the present invention is also a kit suitable for implementing the process of the invention. This kit comprises at least two compartments:
a first compartment comprising a first composition (A) which comprises one or more thiol-comprising reducing agents as defined above, at a pH of inclusively between 1 and 5, preferentially between 2.5 and 4,
a second compartment comprising a second composition (B) which comprises one or more non-thiol-comprising reducing agents as defined above.

The compositions of this kit are packaged in separate compartments, which may be optionally accompanied by suitable identical or different application means, such as fine brushes, coarse brushes or sponges.

The abovementioned kit may also be equipped with means for dispensing the desired mixture on the hair, such as, for instance the device described in patent FR 2 586 913.

The examples that follow illustrate the present invention, and should not in any way be considered as limiting the invention.

### EXAMPLES

### Example 1

a) Protocol for preparing the compositions used in the process of the invention:

Thiolactic acid is tested comparatively in solution at 8% by weight, 4% by weight and 1% by weight in water at pH 3.5.

Para-hydroxyphenol is tested in solution at 5% by weight and 1% by weight in water or in a water/ethanol (75/25 vol) mixture at pH 3.5.

b) Protocol for evaluating the technical effect (stability, performance during/after application, texture)

b1) Each composition prepared in the previous preparation protocol a) was tested on natural and/or bleached locks according to straightening treatment protocols.

b2) Protocol for Brazilian straightening with conventional iron:

The locks of keratin fibres were combed, then were subjected to 10 successive blow drying passes, at position 2 (80° C.) of the hairdryer, with a round brush of medium diameter. Each of the locks was then separated into 2. Each part was subjected to 10 passes with the straightening iron using a comb on each half, then ⅔ passes over all of it at 230° C.

The locks were then washed with a mild shampoo, and dried.

c) Sensory results of the evaluation in vitro, ex vivo

c1/The in vitro tests were carried out on the basis of the following 5 criteria: immediate straightening, feel, manageability, nauseating odour.

The performance qualities of the various compositions treated with the straightening process of the invention were combined in the table below:

| Process | Grade regarding the property | | | |
|---|---|---|---|---|
| of the | “Immediate | | | |
| invention | straightening” | “Feel” | “Manageability” | “Bad odour” |
| G1 | +++ | ++ | + | +++ |
| G2 | +++ | +++ | +++ | + |
| G3 | +++ | ++ | ++ | ++ |
| Negative | — | — | — | — |
| control | | | | |

Each of the above properties was evaluated by experts who assigned a grade ranging from 1 to 4 according to the following scale:

1—no effect (−)

2—slight effect (+)

3—marked effect (++)

4—significant effect (+++)

FIG. 1 is a photograph of the locks of hair obtained.

T represents the control before treatment for straightening.

G1 represents a lock of hair that was treated using a straightening process by means of an aqueous solution of thiolactic acid at 8% by weight and at pH=3.5. The application protocol consists in applying thio lactic acid for 30 min, rinsing, blow drying, applying an iron at 230° C. and then applying H₂O₂.

G2 represents a lock of hair that was treated using a straightening process by means of a solution of thiolactic acid at 1% by weight and at pH=3.5 and of a composition containing para-hydroxy phenol at 5% by weight in a water/ethanol (75/25 vol/vol) solution.

G3 represents a lock of hair that was treated using a straightening process by means of a solution ofthiolatic acid at 4% by weight and at pH=3.5 and of a composition containing para-hydroxy phenol at 1% by weight in a water/ethanol (75/25 vol/vol) solution.

For G2 and G3, the application protocol consists in applying thio lactic acid for 30 min, without rinsing, in applying para-hydroxy phenol for 20 min without rinsing, in blow drying, and then in applying an iron at 230° C.

These tests on the locks show that the compositions of conditions G2 and G3 containing a low amount of thiolactic acid give improved results in terms of hair straightening compared with a composition containing a large amount of thioglycolic acid.

c2/ex vivo tests

The ex vivo tests were carried out on hair taken from Brazilian volunteers, by means of the process of the invention comprising an aqueous composition containing thiolactic acid at 1% by weight and at pH=3.5 and a composition containing para-hydroxy phenol at 5% by weight in water, and then evaluated on the basis of the following 3 criteria: Immediate straightening, feel and manageability.

| | Grade regarding | Grade | Grade |
|---|---|---|---|
| Process | “immediate | regarding | regarding |
| of the | straightening” | “feel” | “manageability” |
| invention | property | property | property |
| Volunteer 1 | +++ | ++ | +++ |
| Volunteer 2 | +++ | ++ | +++ |
| Volunteer 3 | +++ | ++ | +++ |

Each of the above properties was evaluated by experts who assigned a grade ranging from 1 to 4 regarding the gain of the property compared with the initial hair of the volunteer without treatment, according to the following scale:

1—no gain (−)

2—slight gain (+)

3—marked gain (++)

4—significant gain (+++)

The results on hair show a clear hair-straightening effect that can be observed when the process of the invention containing a low amount of non-thiol-comprising reducing agent in a composition at a pH of between 1 and 5 is used (see FIG. 2).

d) Characterizations

d1) Amino acids analysis:

The method for ascertaining the state of the disulfides consists in treating the hair with a composition of iodoacetic acid in order to block the thiol functions of the hair, then digesting the hair using a very acidic treatment, then carrying out a chromatographic analysis of the amino acids. An assessment is then carried out in order to visualize the change in the amino acids before and after treatment. The following are in particular studied: cystine (representing the disulfides), cysteine (reduced disulfides), cysteic acid (super-oxidized disulfides), lanthionine.

The amino acids analysis results show that the treatment with a composition containing an aqueous composition containing thiolactic acid at 1% by weight at a pH of between 1 and 5 does not create any cysteic acid and the amount of disulfide bridges in the fibre is maintained, contrary to what happens when an aqueous composition of thiolactic acid at 8% by weight and at pH=3.5 is used.

The use of an aqueous composition containing 1% of thiolactic acid compared with a composition containing 8% of thio lactic acid thus guarantees the integrity of the keratin fibre.

97% of the natural disulfides of the hair are in fact found after treatment according to the process of the invention, compared with only 80% with the reference treatment using a composition containing 8% of thio lactic acid.

e) Examples of compositions and protocols

The following compositions were prepared with the following ingredients (amount per 100 g of composition % g).

### Compositions Comprising Thiol-Comprising Reducing Agents

| | Composition | Composition | Composition |
|---|---|---|---|
| | 8% thiol- | 4% thiol- | 1% thiol- |
| | comprising | comprising | comprising |
| Ingredients | reducing agent | reducing agent | reducing agent |
| pure | 8.0 | 4.0 | 1.0 |
| thiolactic | | | |
| acid | | | |
| (Aldrich) | | | |
| 2-amino-2- | 3.0 | 1.5 | 0.4 |
| methyl-1- | | | |
| propanol | | | |
| Water | Qs 100 g | Qs 100 g | Qs 100 g |

### Compositions Comprising Non-Thiol-Comprising Reducing Agents

| | | Composition non- |
|---|---|---|
| | Composition non- | thiol-comprising |
| | thiol-comprising | reducing agent in |
| | reducing agent | 25% aqueous |
| Ingredients | formula | alcoholic medium |
| 2-amino-2-methyl-1- | 1 | — |
| propanol (qs pH 3.5) | | |
| Para-hydroxyphenol | 5 | 5 |
| Hydrochloric acid | | 2 |
| 1N | | |
| (qs pH 3.5) | | |
| Ethanol | — | 23.5 |
| Water | Qs 100 g | Qs 100 g |

### Example 2: Treatments of Bleached Caucasian Natural Type Locks SA40

The locks treated are locks of 1 gram of bleached Caucasian natural type which have a reducing solubility of 40; i.e. very damaged locks.

The three treatments described hereunder were respectively performed on three locks.

Composition 1: Solution of thiolactic acid at 1% by weight at pH 3.5.

In a 150 ml flask, 1 gram of thiolactic acid was added, then 50 grams of water was added and then 2-amino-2-methyl-1-propanol was added until a pH of 3.5 was obtained. It was then supplemented with water up to 100 grams and the final pH of composition 1 was checked (pH=3.52)

Composition 2: Solution of para-hydroxyphenol (PHP) at 5% by weight at pH 3.5.

In a 150 ml flask, 5 grams of para-hydroxyphenol was added, then 50 grams of water was added, and then a hydrochloric acid solution 1N was added drop by drop until a pH of 3.5 was obtained. It was then supplemented with water up to 100 grams and the final pH of composition 2 was checked (pH=3.51)

Treatment 1:

A lock of 1 gram is placed flat on an aluminum foil of a lock plate at 27° C. 2 grams of composition 1 were gently deposited along the lock and then applied with a dyeing brush so as to have a homogeneous treatment. It was then left for 30 minutes and then wrung with KIMWIPS paper.

A blow-dry was then carried out at 80° C. (10 passes), then a smoothing with smoothing iron was carried out at 230° C. (10 passes in 6 seconds). Shampoo was then performed.

Treatment 2:

A lock of 1 gram is placed flat on an aluminum foil of a lock plate at 27° C. 2 grams of composition 2 were gently deposited along the lock and then applied with a dyeing brush so as to have a homogeneous treatment. It was then left for 15 minutes and then wrung with KIMWIPS paper.

A blow-dry was then carried out at 80° C. (10 passes), then a smoothing with smoothing iron was carried out at 230° C. (10 passes in 6 seconds). Shampoo was then performed.

Treatment 3:

A lock of 1 gram is placed flat on an aluminum foil of a lock plate at 27° C. 2 grams of composition 1 were gently deposited along the lock and then applied with a dyeing brush so as to have a homogeneous treatment. It was then left for 30 minutes and then wrung with KIMWIPS paper.

Then, 2 grams of composition 2 were gently deposited along the lock and then applied with a dyeing brush so as to have a homogeneous treatment. It was then left for 15 minutes and then wrung with KIMWIPS paper.

A blow-dry was then carried out at 80° C. (10 passes), then a smoothing with smoothing iron was carried out at 230° C. (10 passes in 6 seconds). Shampoo was then performed.

Results

Spectrocolorimetric Evaluation:

The colour of the locks was evaluated in the CIE L* a* b* system using a Minolta Spectrophotometer CM361 Od colorimeter. In this L* a* b* system, the three parameters denote, respectively, the colour intensity (L*), the green/red colour axis (a*) and the blue/yellow colour axis (b*).

The lower the value of L, the darker or more intense the color. The higher the value of a* the hue is red, and the higher the value of b* the hue is yellow.

Build-Up of the Colour or Color Uptake: The variation in colouration between the non-treated bleached Caucasian natural type locks (control) and the treated locks of hair are defined by (ΔE*) according to the following equation:

ΔE*=\sqrt{(L*−Lₒ*)²+(a*−aₒ*)²+(b*−bₒ*)²}

In this equation, L*, a* and b* represent the values measured on locks of hair after treatment, and L₀*, a₀* and b₀* represent the values measured on the non-treated bleached Caucasian natural type locks (control).

The higher the value of ΔE*, the greater the colour build-up.

Chromaticity of the Colour:

The chromaticity is given by the following equation:

C*=\sqrt{(a*)²+(b*)²}

The higher the value of C*, the more chromatic the colour of the keratin fiber.

The results are summarized in the table below:

| | | Build-up of |
|---|---|---|
| | Chromaticity | the colour ΔE* |
| | C* | (D65) |
| Treatment 1 | 28.25 | 5.09 |
| (Comparative) | | |
| Treatment 2 | 27.92 | 6.25 |
| (Comparative) | | |
| Treatment 3 | 34.56 | 12.60 |
| (Invention) | | |

It appears from the above table that the process according to the invention (treatment 3) makes it possible to obtain a colour build-up and a chromaticity that is significantly greater than those obtained with the comparative processes (treatments 1 and 2).

1. Process for shaping and/or straightening keratin fibres, in particular human keratin fibres such as the hair, comprising:
i) the application to said fibres of an acidic composition (A), preferably having a pH of inclusively between 1 and 5 and containing one or more thiol-comprising reducing agents;
ii) the application to said fibres of a distinct composition (B) containing one or more non-thiol-comprising reducing agents, followed
iii) by a step of heat treatment of the fibres by means of a heating tool;
it being understood that steps i) and ii) are carried out separately, i.e. i) then ii) or else ii) then i), preferably in the order i) then ii) and followed by step iii).

2. Process according to claim 1, characterized in that the thiol-comprising reducing agent(s) is (are) chosen from those of formulae i-1 and i-2, and also the organic or mineral acid or base salts thereof, optical isomers thereof and tautomers thereof, and the solvates such as hydrates:
R—SH i-1
R′—S—R″ i-2
In which formulae i-1 and i-2:
R represents
a linear or branched (C₁-C₈)alkyl, preferably (C₁-C₆)alkyl, group which is optionally substituted, preferably substituted, with one or more groups chosen from carboxy C(O)OH, (di)(C₁-C₄)(alkyl)amino, hydroxyl —OH and thiol —SH,
and/or optionally interrupted with one or more heteroatoms or groups chosen from —O—, —S—, —N(R′″)— wherein R″′ represents a hydrogen atom or a linear or branched (C₁-C₄)alkyl group, C(O) or combinations thereof such as —O—C(O)—, —C(O)—O—, —N(R′″)—C(O)— or —C(O)—N(R′″)—;
a (hetero)aryl group optionally substituted in particular with one or more hydroxyl, thiol or carboxy groups;
R′ and R″, which may be identical or different, represent a (C₁-C₈)alkyl group, preferably (C₁-C₆)alkyl group, substituted with one or more groups chosen from hydroxyl, thiol and carboxy;
or else R′ and R″ form, together with the sulfur atom which bears them, a heterocyclic group, comprising from 5 to 7 ring members, which is preferably saturated, which comprises from 1 to 3 heteroatoms, and which is optionally substituted (in particular with one or more (C₁-C₆)alkyl groups optionally substituted with one or more hydroxyl, thiol or carboxy groups), more preferentially the heterocyclic group is a dithiolane group optionally substituted with a (C₁-C₆)alkyl group optionally substituted with one or more carboxy groups.

3. Process according to claim 1, characterized in that the thiol-comprising reducing agent(s) is (are) chosen from those of formula i-1 as defined in the preceding claim, for which R represents a linear or branched (C₁-C₈)alkyl group, preferably (C₁-C₆)alkyl group, substituted with one or more groups chosen from carboxy C(O)OH, amino, hydroxyl —OH, and thiol —SH;
and/or optionally interrupted with one or more heteroatoms or groups chosen from —O—, —N(R′″)— wherein R′″ represents a hydrogen atom or a linear or branched (C₁-C₄)alkyl group, C(O) or combinations thereof such as —O—C(O)—, —C(O)—O—, —N(R′″)—C(O)—, or —C(O)—N(R′″)—, preferably an uninterrupted linear or branched (C₁-C₈)alkyl group, more preferentially (C₁-C₆)alkyl group.

4. Process according to claim 2, characterized in that the thiol-comprising reducing agent(s) is (are) chosen from those of formula i-1 for which R represents:
a phenyl group optionally substituted with one or more hydroxyl, thiol or carboxy groups; or
a heteroaryl comprising from 5 to 10 ring members, which is preferably bicyclic comprising 9 or 10 ring members, comprising from 1 to 4 heteroatoms chosen from O, S or N, preferably N, optionally substituted with one or more hydroxyl or thiol groups.

5. Process according to claim 2, characterized in that the thiol-comprising reducing agent(s) is (are) chosen from those of formula i-2, for which R′ and R″, which may be identical or different, represent a (C₁-C₈)alkyl group, preferably (C₁-C₆)alkyl group, substituted with one or more groups chosen from hydroxyl, thiol, and carboxy.

6. Process according to claim 1, characterized in that the thiol-comprising reducing agent(s) is (are) chosen from thioglycolic acid, thiolactic acid, cysteine, cysteamine, homocysteine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, in particular glyceryl monothioglycolate, and mixtures of these compounds, preferably thioglycolic acid.

7. Process according to claim 1, characterized in that the non-thiol-comprising reducing agent(s) is (are) chosen from ortho-diphenol derivatives and preferably from the compounds of formula (I) 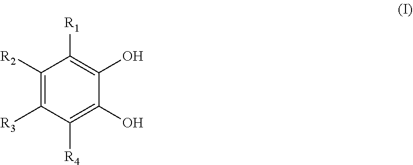
in which formula (I) the substituents:
R₁ to R₄, which may be identical or different, represent:
a hydrogen atom,
a halogen atom,
a hydroxyl radical,
a carboxyl radical;
an alkyl carboxylate or alkoxycarbonyl radical,
an optionally substituted amino radical,
an optionally substituted and linear or branched alkyl radical,
an optionally substituted and linear or branched alkenyl radical,
an optionally substituted cycloalkyl radical,
an alkoxy radical,
an alkoxyalkyl radical,
an alkoxyaryl radical, it being possible for the aryl group to be optionally substituted,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
or two of the substituents carried by two adjacent carbon atoms R₁-R₂, R₂-R₃ or R₃-R₄ form, together with the carbon atoms carrying them, a saturated or unsaturated and aromatic or non-aromatic ring, optionally comprising one or more heteroatoms and optionally fused with one or more saturated or unsaturated rings optionally comprising one or more heteroatoms.

8. Process according to claim 1, characterized in that the non-thiol-comprising reducing agent(s) is (are) chosen from meta-hydroxyphenol derivatives, and preferably from the compounds of formula (II), and also the organic or mineral acid or base salts thereof, and the solvates thereof such as hydrates: 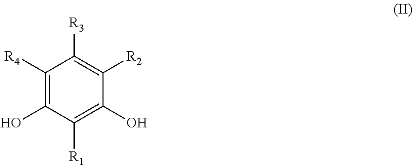
in which formula (II) the substituents:
R₁, R₂ and R₄, which may be identical or different, represent:
a hydrogen atom,
a halogen atom,
a carboxyl radical,
an alkyl carboxylate or alkoxycarbonyl radical,
an optionally substituted amino radical,
an optionally substituted and linear or branched alkyl radical,
an optionally substituted and linear or branched alkenyl radical,
an optionally substituted cycloalkyl radical,
an alkylcarbonyl radical,
a carboxaldehyde radical,
an alkoxy radical,
an alkoxyalkyl radical,
an alkoxyaryl radical, it being possible for the aryl group to be optionally substituted,
an arylalkylcarbonyl radical of which the aryl group, particularly phenyl group, is optionally substituted, preferably with one or more hydroxyl groups,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
R₃ represents:
a hydrogen atom,
a halogen atom,
a hydroxyl radical,
a carboxyl radical,
an alkyl carboxylate or alkoxycarbonyl radical,
an optionally substituted amino radical,
an optionally substituted and linear or branched alkyl radical,
a linear or branched alkenyl radical which is optionally substituted, in particular with a phenyl group which is preferably optionally substituted with one or more (di)(C₁-C₄)(alkyl)amino, or hydroxyl groups,
an optionally substituted cycloalkyl radical,
an alkylcarbonyl radical,
a carboxaldehyde radical,
an alkoxy radical,
an alkoxyalkyl radical,
an alkoxyaryl radical, it being possible for the aryl group to be optionally substituted,
an aryl radical,
a substituted aryl radical,
a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups,
a radical containing one or more silicon atoms,
or two of the substituents borne by two adjacent carbon atoms R₂-R₃ or R₃-R₄ form, together with the carbon atoms carrying them, a saturated or unsaturated and non-aromatic ring, optionally comprising one or more heteroatoms and optionally fused with one or more saturated or unsaturated rings optionally comprising one or more heteroatoms;
particularly, R₁, R₂ and R₄ represent a hydrogen atom or an optionally substituted (C₁-C₆)alkyl group, preferably hydrogen, and/or R₃ represents a hydrogen atom or a linear or branched (C₁-C₆)alkyl or (C₂-C₆)alkenyl group, such as ethenyl, optionally substituted with an aryl group, such as a phenyl group, which is optionally substituted, preferably with one or more (di)(C₁-C₄)(alkyl)amino, or hydroxyl groups;
more particularly, R₁, R₂ and R₄ represent a hydrogen atom or a (C₁-C₄)alkyl group, preferably hydrogen, and R₃ represents a hydrogen atom or an Ar—CH═CH— group with Ar representing an aryl group, in particular a phenyl group, said aryl group being optionally substituted, preferably with one or more hydroxyl groups; in particular said hydroxyl group(s) is (are) substituted in the ortho or para position with respect to the phenyl group.

9. Process according to claim 1, characterized in that the non-thiol-comprising reducing agent(s) is (are) chosen from para-hydroxyphenol derivatives, and preferably from the compounds of formula (III), and also the organic or mineral acid or base salts thereof, and the solvates thereof such as hydrates: 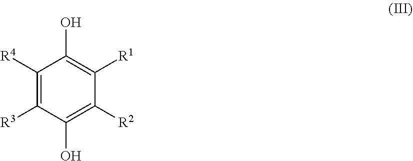
in which formula (III) R¹ to R⁴, which may be identical or different, are a hydrogen atom or an optionally substituted (C₁-C₄)alkyl group, preferably hydrogen.

10. Process according to claim 1, characterized in that the non-thiol-comprising reducing agent(s) is (are) chosen from catechol, gallic acid, para-hydroxyphenol, resveratrol and mixtures thereof.

11. Process according to claim 1, characterized in that the thiol-comprising reducing agent(s) represent(s) from 0.02% to 5% by weight and preferably from 0.1% to 3% by weight relative to the total weight of the composition (A).

12. Process according to claim 1, characterized in that the non-thiol-comprising reducing agent(s) represent(s) from 1% to 10% by weight and preferably from 2% to 8% by weight relative to the total weight of the composition (B).

13. Process according to claim 1, characterized in that the weight ratio between the amount of thiol-comprising reducing agent(s) and the amount of non-thiol-comprising reducing agent(s) is between 0.01 and 10, particularly inclusively between 0.1 and 5, and more preferentially inclusively between 0.2 and 1 in the composition containing them.

14. Process according to claim 1, characterized in that it comprises a step of rinsing the keratin fibres after step i) and/or step ii) and before the heat treatment step (iii).

15. Process according to claim 1, characterized in that it comprises:
i) the application to the fibres of a composition (A) comprising said thiol-comprising reducing agent(s), then
ii) the application to the fibres of a composition (B) comprising said non-thiol-comprising reducing agent(s), then
an optional step of rinsing the fibres, then
iii) a step of heat treatment of the fibres by means of a heating tool.

16. Process according to claim 1, characterized in that the heat treatment step iii) is carried out by means of a heating tool chosen from a hairstyling hood, a straightening iron, a hairdryer and an infrared-ray dispenser, preferably a straightening iron.

17. Process according to claim 1, characterized in that the heat treatment step is carried out at a temperature ranging from 30 to 250° C., preferably from 60 to 230° C. and more preferentially from 100 to 150° C.

18. Process according to claim 1, characterized in that it does not use hydrogen peroxide, more preferably the process does not use any chemical oxidizing agent other than oxygen of the air.

19. Kit comprising at least two compartments:
a first compartment comprising a first acidic composition (A) which comprises one or more thiol-comprising reducing agents,
a second compartment comprising a second composition (B) which comprises one or more non-thiol-comprising reducing agents.

20. Cosmetic composition, comprising:
i) from 0.02% to 5% by weight, preferably from 0.1% to 3% by weight, relative to the total weight of the composition, of one or more thiol-comprising reducing agents chosen from thiolactic acid, cysteamine, homocysteine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, in particular glyceryl monothioglycolate, and mixtures of these compounds;
it being understood that the composition is acid, in particular has a pH of inclusively between 1 and 5, preferably between 2.5 and 4.

21. Cosmetic composition according to claim 20, characterized in that it does not comprise hydrogen peroxide, more preferably the cosmetic composition does not comprise any chemical oxidizing agent other than oxygen in the air.
